SEQUENCE LISTING (6 sequences)

SEQ ID NO: 1 - DNA, 516 bp, Ovis aries
tgctacctgtcgcgaaaactgatgctggacgctcgagaaaatttaaaactgctggaccgtatgaatcgattgtctccgcacagctgcctgcaagaccggaaagacttcggtctgccgcaggaaatggttgaaggtgaccaactgcaaaaagaccaagctttcccggtactgtatgaaatgctgcagcagtctttcaacctgttctacactgaacattcttcggccgcttgggacactactcttctagaacaactgtgcactggtctgcaacagcaactggaccatctggacacttgccgtggccaggttatgggtgaagaagactctgaactgggtaacatggatccgatcgttactgttaaaaaatatttccagggtatctacgactacctgcaggaaaaaggttactctgactgcgcttgggaaatcgtacgcgttgaaatgatgcgggccctgactgtgtcgactactctgcaaaaacggttaactaaaatgggtggtgacctgaattctccg

SEQ ID NO: 2 - protein, 172 aa, Ovis aries
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 3 - protein, 172 aa, Artificial
  recombinant IFNtau based on Ovis aries sequence
CYLSERLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 4 - DNA, 516 bp, Artificial
  recombinant IFN-tau based on Ovis aries sequence
tgctacctgtcggagcgactgatgctggacgctcgagaaaatttaaaactgctggaccgtatgaatcgattgtctccgcacagctgcctgcaagaccggaaagacttcggtctgccgcaggaaatggttgaaggtgaccaactgcaaaaagaccaagctttcccggtactgtatgaaatgctgcagcagtctttcaacctgttctacactgaacattcttcggccgcttgggacactactcttctagaacaactgtgcactggtctgcaacagcaactggaccatctggacacttgccgtggccaagttatgggtgaagaagactctgaactgggtaacatggatccgatcgttactgttaaaaaatatttccagggtatctacgactacctgcaggaaaaaggttactctgactgcgcttgggaaatcgtacgcgttgaaatgatgcgggccctgactgtgtcgactactctgcaaaaacggttaactaaaatgggtggtgacctgaattctccg

SEQ ID NO: 5 - protein, 295 aa, Homo sapiens [sequence omitted]

SEQ ID NO: 6 - protein, 186 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

(19) United States
(12) Patent Application Publication
(10) Pub. No.: US 2005/0226845 A1
(43) Pub. Date: Oct. 13, 2005

(54) Method of treatment using interferon-tau

(76) Inventors: Chih-Ping Liu, San Francisco, CA (US); Lorelie H. Villarete, Alameda, CA (US)

Correspondence Address: PERKINS COIE LLP, P.O. BOX 2168, MENLO PARK, CA, 94026, US

(21) Appl. No.: 11/040,706

(22) Filed: Jan. 21, 2005

Related U.S. Application Data
(63) Continuation-in-part of application No. 10/884,741, filed on Jul. 2, 2004.
Application No. 10/884,741 is a continuation-in-part of application No. 10/824,710, filed on Apr. 14, 2004.
(63) Continuation-in-part of application No. 10/825,068, filed on Apr. 14, 2004.
(63) Continuation-in-part of application No. 10/825,382, filed on Apr. 14, 2004.
Application No. 10/825,382 is a continuation-in-part of application No. 10/825,457, filed on Apr. 14, 2004.
(60) Provisional application No. 60/552,279, filed on Mar. 10, 2004.
(60) Provisional application No. 60/552,279, filed on Mar. 10, 2004.

(51) Int. Cl. A61K 38/21

(52) U.S. Cl. 424/85.4

(57) ABSTRACT

Methods of treating a disease or condition responsive to interleukin-10 therapy in a mammal are provided. In one form, a method includes orally administering a therapeutically effective amount of interferon tau to the mammal. In other forms of the invention, the method includes administering a second therapeutic agent to the mammal in addition to interleukin-10 either simultaneously or sequentally.

### CROSS-REFERENCE TO RELATED APPLICATIONS

This application is a continuation-in-part of U.S. patent application Ser. No. 10/884,741, filed Jul. 2, 2004, which is a continuation-in-part of U.S. patent application Ser. No. 10/824,710, filed Apr. 14, 2004, and which claims the benefit of U.S. Provisional Patent Application Ser. No. 60/552,279, filed Mar. 10, 2004. This application is also a continuation-in-part of U.S. patent application Ser. Nos. 10/825,068; 10/825,382; and 10/825,457, all of which were filed on Apr. 14, 2004, and all of which claim the benefit of U.S. Provisional Patent Application Ser. No. 60/552,279, filed Mar. 10, 2004.

### FIELD OF THE INVENTION

The present invention relates to pharmaceutical compositions containing interferon-tau and methods of uses thereof. More particularly, the invention relates to methods of treating diseases or conditions responsive to interleukin-10 (IL-10) therapy in a mammal by administering interferon-tau (IFNτ) either alone or in combination with one or more therapeutic agents.

### BACKGROUND OF THE INVENTION

Interferon-tau (hereinafter “IFNτ” or “interferon-τ”) was discovered originally as a pregnancy recognition hormone produced by the trophectoderm of ruminant conceptuses (Imakawa, K. et al, Nature, 330:377-379, (1987); Bazer, F. W. and Johnson, H. M., Am. J. Repro. Immunol, 26:19-22, (1991)). The distribution of the IFNτ gene is restricted to ruminants, including cattle, sheep, and goats, (Alexenko, A. P. et al., J. Interferon and Cytokine Res., 19:1335-1341, (1999)) but has been shown to have activity in cells belonging to other species including humans and mice (Pontzer, C. H. et al., Cancer Res., 51:5304-5307, (1991); Alexenko, A. P. et al., J. Interferon and Cytokine Res., 20:817-822, (2000)). For example, IFNτ has been demonstrated to possess antiviral, (Pontzer, C. H. et al., Biochem. Biophys. Res. Commun., 152:801-807, (1988)), antiproliferative, (Pontzer, C. H., et al., 1991) and immunoregulatory activities (Assal-Meliani, A., Am. J. Repro. Immunol., 33:267-275 (1995)).

While IFNτ displays many of the activities classically associated with type I IFNs, such as interferon-α and interferon-β, considerable differences exist between IFNτ and the other type I IFNs. The most prominent difference is the role of IFNτ in pregnancy in ruminant species. The other IFNs have no similar activity in pregnancy recognition. Also different is viral induction. All type I IFNs, except IFNτ, are induced readily by virus and dsRNA (Roberts, et al., Endocrine Reviews, 13:432 (1992)). Induced IFN-α and IFN-β expression is transient, lasting approximately a few hours. In contrast, IFNτ synthesis, once induced, is maintained over a period of days (Godkin, et al., J. Reprod. Fert., 65:141 (1982)). On a per-cell basis, 300-fold more IFNτ is produced than other type I IFNs (Cross, J. C. and Roberts, R. M., Proc. Natl. Acad. Sci. USA 88:3817-3821 (1991)).

Another difference lies in the amino acid sequences of IFNτ and other type I interferons. The percent amino acid sequence similarity between the interferons α_{2b}, β₁, ω₁, γ, and τ are summarized in the table below.

| | rHuIFNα_{2b} | rHuIFNβ₁ | rHuIFN₁ω₁ | rHuIFN_{γ} | rOvIFNτ |
|---|---|---|---|---|---|
| rHuIFNα_{2b} | | 33.1 | 60.8 | 11.6 | 48.8 |
| rHuIFNβ₁ | 33.1 | | 33.1 | 12.2 | 33.8 |
| rHuIFNω₁ | 60.8 | 33.1 | | 10.2 | 54.9 |
| rHuIFN_{γ} | 11.6 | 12.2 | 10.2 | | 10.2 |
| rOvIFNτ | 48.8 | 33.8 | 54.9 | 10.2 | |

Sequence comparison determined from the following references:
Taniguchi et al., Gene, 10(1): 11 (1980).
Adolf et al., Biochim. Biophys. Acta, 1089(2): 167 (1991).
Streuli et al., Science, 209: 1343 (1980).
Imakawa et al., Nature, 330: 377 (1987).

Recombinant ovine IFNτ is 48.8 percent homologous to IFNα_{2b} and 33.8 percent homologous to IFNβ₁. Because of this limited homology between IFNτ and IFNα and between IFNτ and IFNβ, it cannot be predicted whether or not IFNτ would behave in the same manner as IFNα or IFNβ when administered orally. IFNτ is also reported to have a low receptor binding affinity for type I receptors on human cells (Brod, S., J. Interferon and Cytokine Res., 18:841 (1999); Alexenko, A. et al., J. Interferon and Cytokine Res., 17:769 (1997)). Additionally, the fact that IFNτ is a non-endogeneous human protein generates the potential for systemic neutralizing antibody formation when IFNτ is injected into the human body (Brod, S., J. Interferon and Cytokine Res., 18:841 (1999). These differences between IFNτ and the other interferons make it difficult to predict whether IFNτ will provide a therapeutic benefit when administered to a human. Teachings in the art relating to oral administration of IFNα, IFNβ, or any other non-tau interferon, fail to provide a basis for drawing any expectations for IFNτ.

One limiting factor in the use of IFNτ, as well as proteins and polypeptides in general, is related to biodistribution, as affected by protein interaction with plasma proteins and blood cells, when given parenterally. The oral route of administration is even more problematic due to proteolysis in the stomach, where the acidic conditions can destroy the molecule before reaching its intended target. For example, polypeptides and protein fragments, produced by action of gastric and pancreatic enzymes, are cleaved by exo- and endopeptidases in the intestinal brush border membrane to yield di- and tri-peptides. If proteolysis by pancreatic enzymes is avoided, polypeptides are subject to degradation by brush border peptidases. Polypeptides or proteins that might survive passage through the stomach are subject to metabolism in the intestinal mucosa where a penetration barrier prevents entry into cells. For this reason, much effort has been focused on delivering proteins to the oral-pharyngeal region in the form of a lozenge or solution held in the oral cavity for a period of time.

The role of cytokines in various diseases and correlations between cytokine blood levels with disease onset and severity is of interest to the medical community. For example, it has been shown or suggested that the following diseases may be benefited by interleukin-10 therapy or otherwise show some linkage between interleukin-10 and the particular disease or condition: liver fibrosis [Nelson, D. R. et al. Hepatology 38(4):859-868 (2003); Louis, H., Acta Gastr. Belg. 66(1):7-14 (2003)]; pulmonary fibrosis [Aral, T., et al., Am. J. Physiol. Lung. Cell. Mol. Physiol. 278:L914-L922 (2000)]; Alzheimer's disease [De Luigi, A. et al., Mech. Age. Dev. (16):1985-1995 (2001); Remarzue, E. J., and Bollen, E. L., Exp. Gerontol. 36(1):171-176 (2001); Town, T. et al., J. Neuroimmunol. 132(1-2):49-59 (2002)]; stroke [Frenkel, D. et al., J. Immunol. 171(12):6549-6555 (2003)]; anti-phospholipid syndrome [Krause, I, et al. Eur. J. Immunol. 32(12):3414-3424 (2002)]; atherosclerosis [Ohashi R. et al., Med Sci Monit 10(11):RA255-60 (2004); Zimmerman M A, et al., J Surg Res 121(2):206-13 (2004); Fichtlscherer S, et al., J Am Coll Cardiol 44(1):44-9 (2004); Potteauz S, et al., Arterioscler Thromb Vasc Biol 24(8):1474-8 (2004)]; rejection from organ transplantation [Zheng H X, et al., J Heart Lung Transplant 23:541-6 (2004); Fischer S, et al., J Thora Cardiovas Surg 126:1174-80 (2003); Sembeil R, et al., Transpl Immunol 13(1):1-8 (2004)];autism [Jyonouchi, H., et al., J. Neuroimmun. 120:170-179 (2001)]; chronic obstructive pulmonary disease [Takanashi, S., et al., Eur. Respir. J. 14:309-314 (1999); various autoimmune disorders, including type I diabetes mellitus [Slavin A J, et al., Int Immunol 13(6):825-33 (20010; Zhang Z L, et al., Acta Pharmacol Sin 24(8):751-6 (2003)]; arthritis, including rheumatoid arthritis [Tanaka Y, et al., Inflamm Res 45(6):283-8 (1996); Detanico T, et al., Clin Exp Immunol 135(2):336-42 (2004); Driessler F, et al., Clin Exp Immunol 135(1):64-73 (2004)], psoriasis [Asadullah K, et al., Pharmacol Rev 55(2):241-69 (2003); Asadullah K, et al., Curr Drug Targets Inflamm Allergy 3(2):185-92 (2004)] multiple sclerosis (Soos et al., J Neuroimmunol 75:43-50 (1997)]; uveitis [Kezuka, T., et al., J. Immunol. 173(2):1454-1462 (2004), Sun, B., et al., Exp. Eye Res. 70:493-502 (2000); allergies [Zuany-Amorim, C. et al., J. Clin. Invest. 95:2644-2651 (1995), Borish, L., et al., J. Allergy Clin. Immunol 97:1288-1296 (1996)]; inflammatory bowel disease [Li M C and He S H, World J Gastroenterol 10(5):620-5 (2004); Braat H, et al., Expert Opin Biol Ther 3(5):725-31 (2003)], and optic neuritis [Navikas, V., et al., Scand. J. Immunol. 41(2):171-178 (1995)]. Although many of these diseases or conditions may be improved or otherwise treated with various methods and compositions, many of such methods and compositions have drawbacks such that there is a continuing need for safe and effective methods and compositions to treat such diseases or conditions. The present invention addresses this need.

### SUMMARY OF THE INVENTION

Interleukin 10 has been found to be associated with a wide variety of disease states. Accordingly, methods of treating a disease or condition responsive to interleukin-10 therapy in a mammal are provided.

In one aspect of the invention, a method of treating a disease or condition responsive to interleukin-10 therapy in a mammal includes orally administering a daily dosage of greater than about 5×10⁸ Units of interferon tau to the mammal.

The method may be amenable for treating a wide variety of diseases or conditions, including Alzheimer's disease, liver fibrosis, pulmonary fibrosis, optic neuritis, stroke, atherosclerosis, anti-phospholipid syndrome, chronic obstructive pulmonary disease, autism, rejection from organ transplantation and a wide variety of autoimmune disorders.

In certain forms of the invention, the method includes administering a second therapeutic agent to the mammal in addition to interleukin-10 either simultaneously or sequentially. Such combination therapy can result in additive or synergic improvements in ameliorating the disease or condition, including ameliorating the progression and/or to facilitate resolution of the condition.

It is an object of the invention to provide methods of treating a disease or condition responsive to interleukin-10 therapy in a mammal.

These and other objects and features of the invention will be more fully apparent from the description herein.

### BRIEF DESCRIPTION OF THE SEQUENCES

SEQ ID NO:1 is the nucleotide sequence of a synthetic gene encoding ovine interferon-tau (IFNτ).

SEQ ID NO:2 corresponds to an amino acid sequence of mature ovine interferon-τ (IFNτ; oTP-1; GenBank Accession No. Y00287; PID g1358).

SEQ ID NO:3 corresponds to an amino acid sequence of mature ovine IFNτ, where the amino acid residues at positions 5 and 6 of the sequence are modified relative to the sequence of SEQ ID NO:2.

SEQ ID NO:4 is a synthetic nucleotide sequence encoding the protein of SEQ ID NO:3.

SEQ ID NO:5 corresponds to an amino acid sequence of human myelin oligodendrocyte protein (GenBank Accession No. BC035938).

SEQ ID NO:6 corresponds to an amino acid sequence of human myelin basic protein (GenBank Accession No. NM_{—}002385).

### DESCRIPTION OF THE PREFERRED EMBODIMENT

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present invention provides methods for treating various diseases or conditions with interferon-tau, either alone or in combination with other therapeutic agents. More specifically, the present invention provides methods for treating conditions responsive to interleukin-10 therapy, including Alzheimer's disease, liver fibrosis, lung fibrosis, anti-phospholipid syndrome, optic neuritis, stroke, atherosclerosis, autism, chronic obstructive pulmonary disease, rejection from organ transplantation and several autoimmune disorders. In one form, a method includes administering to a mammal in need thereof a therapeutically effective amount of interferon-tau, including a daily dosage of greater than about 5×10⁸ Units. As discussed above, these disease states or conditions may be amenable to treatment with interleukin-10. Although not being limited by theory, as interferon-tau is effective in up-regulating the blood level of interleukin-10, treatment with interferon-tau may be effective in decreasing the severity of the disease or condition or may otherwise eliminate the disease or condition.

In yet other forms of the invention, interferon-tau may be administered with a second therapeutic agent. The combination therapy may allow for either an additive or synergistic interaction of interferon-tau and the second therapeutic agent to achieve a decrease in the severity of the disease or condition, or otherwise improve the disease or condition, to an extent greater than either agent alone could achieve.

In one aspect of the invention, methods for treating a disease or condition responsive to interleukin-10 therapy are provided. The method may be used to treat a disease or condition other than a viral disease or condition, including, for example, a disease or condition other than one caused by a Hepatitis C viral infection. In one form, a method includes administering to a mammal a therapeutically effective amount of interferon-tau, such as a daily dosage of greater than about 5×10⁸ Units.

### I. DEFINITIONS

Interferon-tau, abbreviated as IFNτ or interferon-τ, refers to any one of a family of interferon proteins having at least one characteristic from each of the following two groups of characteristics: (i) (a) anti-luteolytic properties, (b) anti-viral properties, (c) anti-cellular proliferation properties; and (ii) about 45 to 68% amino acid homology with α-interferons and greater than 70% amino acid homology to known IFNτ sequences (e.g., Ott, et al., J. Interferon Res., 11:357 (1991); Helmer, et al., J. Reprod. Fert., 79:83 (1987); Imakawa, et al., Mol. Endocrinol, 3:127 (1989); Whaley, et al., J. Biol. Chem., 269:10846 (1994); Bazer, et al., WO 94/10313 (1994)). Amino acid homology can be determined using, for example, the LALIGN program with default parameters. This program is found in the FASTA version 1.7 suite of sequence comparison programs (Pearson and Lipman, PNAS, 85:2444 (1988); Pearson, Methods in Enzymology, 183:63 (1990); program available from William R. Pearson, Department of Biological Chemistry, Box 440, Jordan Hall, Charlottesville, Va.). IFNτ sequences have been identified in various ruminant species, including but not limited to, cow (Bovine sp., Helmer S. D., J. Reprod. Fert., 79:83 (1987); Imakawa, K., Mol. Endocrinol., 119:532 (1988)), sheep (Ovine sp.), musk ox (Ovibos sp.), giraffe (Giraffa sp., GenBank Accession no. U55050), horse (Equus caballus), zebra (Equus burchelli, GenBank Accession no. NC005027), hippopotamus (Hippopotamus sp.), elephant (Loxodonta sp.), llama (Llama glama), goat (Capra sp., GenBank Accession nos. AY357336, AY357335, AY347334, AY357333, AY357332, AY357331, AY357330, AY357329, AY357328, AY357327), and deer (Cervidae sp.). The nucleotide sequences of IFNτ for many of these species are reported in public databases and/or in the literature (see, for example, Roberts, R. M. et al., J. Interferon and Cytokine Res., 18:805 (1998), Leaman D. W. et al., J. Interferon Res., 12:1 (1993), Ryan, A. M. et al., Anim. Genet., 34:9 (1996)). The term “interferon-tau” intends to encompass the interferon-tau protein from any ruminant species, exemplified by those recited above, that has at least one characteristic from each of the following two groups of characteristics listed above.

Ovine IFNτ (OvIFNτ) refers to a protein having the amino acid sequence as identified herein as SEQ ID NO:2, and to proteins having amino acid substitutions and alterations such as neutral amino acid substitutions that do not significantly affect the activity of the protein, such as the IFNτ protein identified herein as SEQ ID NO:3. More generally, an ovine IFNτ protein is one having about 80%, more preferably 90%, sequence homology to the sequence identified as SEQ ID NO:2. Sequence homology is determined, for example, by a strict amino acid comparison or using one of the many programs commercially available.

Treating a condition refers to administering a therapeutic substance effective to reduce the symptoms of the condition and/or lessen the severity of the condition.

Oral refers to any route that involves administration by the mouth or direct administration into the stomach or intestines, including gastric administration.

Intestine refers to the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

“Measurable increase in blood IL-10 lever” refers to a statistically meaningful increase in blood (serum and/or blood-cell) levels of interleukin-10, typically at least a 20% increase, more preferably at least a 25% increase, over pre-treatment levels measured under identical conditions. Methods for measuring IL-10 levels in the blood are described herein using a commercially-available enzyme-linked immunosorbent assay (ELISA) kit. A fold-increase is determined by dividing the value at timepoint x by the screening or baseline value. A percent increase is determined by finding the difference between the value at timepoint x and the screening or baseline value; dividing this difference by the screening or baseline value; and multiplying the quotient by 100.

“A “daily dosage of greater than 5×10⁸ Units” refers to an amount of IFNτ sufficient to provide more than about 5×10⁸ antiviral Units of protein, where the antiviral activity of IFNτ is measured using a standard cytopathic effect inhibition assay known in the art. It will be appreciated that the amount (i.e., mg) of protein to provide a daily dosage of greater than 5×10⁸ Units will vary according to the specific antiviral activity of the protein.

### II. INTERFERON-τCOMPOSITIONS AND METHOD OF TREATMENT

A. Interferon-τ

The first IFNτ to be identified was ovine IFNτ (IFNτ), as a 18-19 kDa protein. Several isoforms were identified in conceptus (the embryo and surrounding membranes) homogenates (Martal, J., et al., J. Reprod. Fertil. 56:63-73 (1979)). Subsequently, a low molecular weight protein released into conceptus culture medium was purified and shown to be both heat labile and susceptible to proteases (Godkin, J. D., et al., J. Reprod. Fertil. 65:141-150 (1982)). IFNτ was originally called ovine trophoblast protein-one (oTP-1) because it was the primary secretory protein initially produced by trophectoderm of the sheep conceptus during the critical period of maternal recognition in sheep. Subsequent experiments have determined that IFNτ is a pregnancy recognition hormone essential for establishment of the physiological response to pregnancy in ruminants, such as sheep and cows (Bazer, F. W., and Johnson, H. M., Am. J. Reprod. Immunol. 26:19-22 (1991)).

An IFNτ cDNA obtained by probing a sheep blastocyst library with a synthetic oligonucleotide representing the N-terminal amino acid sequence (Imakawa, K. et al, Nature, 330:377-379, (1987)) has a predicted amino acid sequence that is 45-55% homologous with IFN-αs from human, mouse, rat, and pig and 70% homologous with bovine IFN-αII, now referred to as IFN-Ω. Several cDNA sequences have been reported which may represent different isoforms (Stewart, H. J., et al, Mol. Endocrinol. 2:65 (1989); Klemann, S. W., et al., Nuc. Acids Res. 18:6724 (1990); and Charlier, M., et al., Mol. Cell Endocrinol. 76:161-171 (1991)). All are approximately 1 kb with a 585 base open reading frame that codes for a 23 amino acid leader sequence and a 172 amino acid mature protein. The predicted structure of IFNτ as a four helical bundle with the amino and carboxyl-termini in apposition further supports its classification as a type I IFN (Jarpe, M. A., et al., Protein Engineering 7:863-867 (1994)).

### Overview of the Interferons

| Aspects | Type I | Type I | Type I | Type II |
|---|---|---|---|---|
| Types | α & ω | β | τ | γ |
| Produced by: | leukocyte | fibroblast | trophoblast | lymphocyte |
| Antiviral | + | + | + | + |
| Antiproliferative | + | + | + | + |
| Pregnancy Signaling | − | − | + | − |

While IFNτ displays some of the activities classically associated with type I IFNs (see Table, above), considerable differences exist between it and the other type I IFNs. The most prominent difference is its role in pregnancy, detailed above. Also different is viral induction. All type I IFNs, except IFNτ, are induced readily by virus and dsRNA (Roberts, R. M., et al., Endocrin. Rev. 13:432-452 (1992)). Induced IFN-α and IFN-β expression is transient, lasting approximately a few hours. In contrast, IFNτ synthesis, once induced, is maintained over a period of days (Godkin, et al., 1982). On a per-cell basis, 300-fold more IFNτ is produced than other type I IFNs (Cross, J. C., and Roberts, R. M., Proc. Natl. Acad. Sci. USA 88:3817-3821 (1991)).

Other differences may exist in the regulatory regions of the IFNτ gene. For example, transfection of the human trophoblast cell line JAR with the gene for bovine IFNτ resulted in antiviral activity while transfection with the bovine IFN-Ω gene did not. This implies unique transacting factors involved in IFNτ gene expression. Consistent with this is the observation that while the proximal promoter region (from −126 to the transcriptional start site) of IFNτ is highly homologous to that of IFN-α and IFN-β; the region from −126 to −450 is not homologous and enhances only IFNτ expression (Cross, J. C., and Roberts, R. M., Proc. Natl. Acad. Sci. USA 88:3817-3821 (1991)). Thus, different regulatory factors appear to be involved in IFNτ expression as compared with the other type I IFNs.

The 172 amino acid sequence of ovine-IFNτ is set forth, for example, in U.S. Pat. No. 5,958,402, and its homologous bovine-IFNτ sequence is described, for example, in Helmer et al., J. Reprod. Fert., 79:83-91 (1987) and Imakawa, K. et al., Mol. Endocrinol., 3:127 (1989). The sequences of ovine-IFNτ and bovine-IFNτ from these references are hereby incorporated by reference. An amino acid sequence of ovine IFNτ is shown herein as SEQ ID NO:2.

1. Isolation of IFNτ

IFNτ may be isolated from conceptuses collected from pregnant sheep and cultured in vitro in a modified minimum essential medium as described by Godkin, J. D., et al., J. Reprod. Fertil. 65:141-150 (1982) and Vallet, J. L., et al., Biol. Reprod. 37:1307 (1987). The IFNτ may be purified from the conceptus cultures by ion exchange chromatography and gel filtration. The homogeneity of isolated IFNτ may be assessed by sodium dodecyl sulfate polyacrylamide gel electrophoresis (Maniatis, T., et a., in MOLECULAR CLONING: A LABORATORY MANUAL, Cold Spring Harbor Laboratory, Cold Spring Harbor, N.Y. (1982); Ausubel, F. M., et al., in CURRENT PROTOCOLS IN MOLECULAR BIOLOGY, John Wiley & Sons, Inc., Media, Pa. (1988)), and determination of protein concentration in purified IFNτ samples may be performed using the bicinchoninic (BCA) assay (Pierce Chemical Co., Rockford, Ill.; Smith, P. K., et al., Anal. Biochem. 150:76 (1985)).

2. Recombinant Production of IFNτ

Recombinant IFNτ protein may be produced from any selected IFNτ polynucleotide fragment using a suitable expression system, such as bacterial or yeast cells. The isolation of IFNτ nucleotide and polypeptide sequences is described in PCT publication WO/94/10313, which is incorporated by reference herein.

To make an IFNτ expression vector, an IFNτ coding sequence (e.g, SEQ ID NOS:1 or 4) is placed in an expression vector, e.g., a bacterial expression vector, and expressed according to standard methods. Examples of suitable vectors include lambda gt11 (Promega, Madison Wisc.); pGEX (Smith, P. K. et al., Anal. Biochem. 150:76 (1985)); pGEMEX (Promega); and pBS (Strategene, La Jolla Calif.) vectors. Other bacterial expression vectors containing suitable promoters, such as the T7 RNA polymerase promoter or the tac promoter, may also be used. Cloning of the IFNτ synthetic polynucleotide into a modified pIN III omp-A expression vector is described in the Materials and Methods.

For the studies described herein, the IFNτ coding sequence present in SEQ ID NO:4 was cloned into a vector, suitable for transformation of yeast cells, containing the methanol-regulated alcohol oxidase (AOX) promoter and a Pho1 signal sequence. The vector was used to transform P. pastoris host cells and transformed cells were used to express the protein according to the manufacturer's instructions (Invitrogen, San Diego, Calif.).

Other yeast vectors suitable for expressing IFNτ for use with methods of the present invention include 2 micron plasmid vectors (Ludwig, D. L. et al., Gene, 132:33 (1993)), yeast integrating plasmids (Shaw, K. J. et al., DNA, 7:117 (1988)), YEP vectors (Shen, L. P. et al., Sci. Sin., 29:856 (1986)), yeast centromere plasmids (YCps), and other vectors with regulatable expression (Hitzeman, R. A. et al., U.S. Pat. No.4,775,622, issued Oct. 4, 1988; Rutter, W. J. et al., U.S. Pat. No.4,769,238, issued Sep. 6,1988; Oeda, K. et al., U.S. Pat. No. 4,766,068, issued Aug. 23, 1988). Preferably, the vectors include an expression cassette containing an effective yeast promoter, such as the MFα1 promoter (Bayne, M. L. et al., Gene 66:235-244 (1988), GADPH promoter (glyceraldehyde-3-phosphate-dehydrogenase; Wu, D. A. et al., DNA, 10:201 (1991)) or the galactose-inducible GAL10 promoter (Ludwig, D. L. et al., Gene, 132:33 (1993); Feher, Z. et al., Curr. Genet., 16:461 (1989)); Shen, L. P. et al., Sci. Sin., 29:856 (1986)). The yeast transformation host is typically Saccharomyces cerevisiae, however, as illustrated above, other yeast suitable for transformation can be used as well (e.g., Schizosaccharomyces pombe, Pichia pastoris and the like).

Further, a DNA encoding an IFNτ polypeptide can be cloned into any number of commercially available vectors to generate expression of the polypeptide in the appropriate host system. These systems include the above described bacterial and yeast expression systems as well as the following: baculovirus expression (Reilly, P. R. et al., BACULOVIRUS EXPRESSION VECTORS: A LABORATORY MANUAL, (1992); Beames et al., Biotechniques, 11:378 (1991); Clontech, Palo Alto Calif.); plant cell expression, transgenic plant expression, and expression in mammalian cells (Clontech, Palo Alto Calif.; Gibco-BRL, Gaithersburg Md.). The recombinant polypeptides can be expressed as fusion proteins or as native proteins. A number of features can be engineered into the expression vectors, such as leader sequences which promote the secretion of the expressed sequences into culture medium. The recombinantly produced polypeptides are typically isolated from lysed cells or culture media. Purification can be carried out by methods known in the art including salt fractionation, ion exchange chromatography, and affinity chromatography. Immunoaffinity chromatography can be employed, as described above, using antibodies generated based on the IFNτ polypeptides.

In addition to recombinant methods, IFNτ proteins or polypeptides can be isolated from selected cells by affinity-based methods, such as by using appropriate antibodies. Further, IFNτ peptides (e.g. SEQ ID NOS:2 or 3) may be chemically synthesized using methods known to those skilled in the art.

### III. METHODS OF USE

In a first aspect, the invention provides a method for treating in a mammal, such as a human, a disease or condition responsive to interferon therapy. A condition “responsive to interferon therapy” is one in which the existence, progression, or symptoms of the condition is altered upon administration of an interferon, in particular a type-I interferon, and more particularly, interferon-tau. Conditions responsive to treatment with IFNα or IFNβ may also respond to treatment with IFNτ. More preferably, a condition responsive to interferon therapy is one where the existence, progression, or symptoms of the condition are alleviated by IFNτ administered in an oral or non-oral route, such as injection. The method further includes treating a disease or condition responsive to cytokine therapy, such as interleukin therapy and, for example, interleukin-10 therapy. A disease or condition “responsive to cytokine therapy”, such as interleukin-10 therapy, is one in which the existence, progression, or symptoms of the condition is altered upon administration of a cytokine, such as interleukin-10, or by otherwise up-regulating the amount of the cytokine in the blood of the mammal to be treated. The methods described herein encompass providing IFNτ, preferably in an orally-administrable dosage form for administration to the stomach and/or intestines, in an amount effective for therapy, as evidenced by, for example, an increase in blood IL-10 level determined from studies on similarly situated patients or on the particular individual patient being treated.

A. Diseases and/or Conditions Treated

The disease or condition to be treated is typically one that is responsive to cytokine therapy, such as interleukin-10 therapy. A wide variety of diseases or conditions may be responsive to interleukin-10 therapy and are therefore amenable to treatment with interferon-tau, either alone or in combination with another therapeutic agent. For example, the methods of the invention may be advantageously used to treat neurological disorders, including Alzheimer's disease and autism; fibrotic diseases, including pulmonary fibrosis and liver fibrosis; autoimmune diseases, including anti-phospholipid syndrome, arthritis, allergies, diabetes, such as type I diabetes mellitus; inflammatory bowel disease, including Crohn's disease and ulcerative colitis; psoriasis; multiple sclerosis, uveitis; chronic obstructive pulmonary disease, including chronic bronchitis and emphysema; diseases or conditions characterized by cellular, tissue or organ damage or death due to decreased blood flow and/or oxygen to the cell, tissue or organ, including stroke; and atherosclerosis; and rejection from organ transplant.

B. Formulations

Oral preparations containing IFNτ can be formulated according to known methods for preparing pharmaceutical compositions. In general, the IFNτ therapeutic compositions are formulated such that an effective amount of the IFNτ is combined with a suitable additive, carrier and/or excipient in order to facilitate effective oral administration of the composition. For example, tablets and capsules containing IFNτ may be prepared by combining IFNτ (e.g., concentrated or lyophilized IFNτ protein) with additives such as pharmaceutically acceptable carriers (e.g., lactose, corn starch, microcrystalline cellulose, sucrose, maltitol, glycerol, propylene glycol, Tris, etc.), binders (e.g., alpha-form starch, methylcellulose, carboxymethylcellulose, hydroxypropylcellulose, hydroxypropylmethylcellulose, polyvinylpyrrolidone), disintegrating agents (e.g., carboxymethylcellulose calcium, starch, low substituted hydroxy-propylcellulose), surfactants (e.g., Tween 80, polyoxyethylene-polyoxypropylene copolymer), antioxidants (e.g., L-cysteine, sodium sulfite, sodium ascorbate), lubricants (e.g., magnesium stearate, talc), amino acids (e.g. leucine, alanine, histidine, etc.) or the like.

Further, IFNτ polypeptides of the present invention can be mixed with a solid, pulverulent or other carrier, for example lactose, saccharose, sorbitol, mannitol, starch, such as potato starch, corn starch, millopectine, cellulose derivative or gelatine, and may also include lubricants, such as magnesium or calcium stearate, or polyethylene glycol waxes compressed to the formation of tablets. By using several layers of the carrier or diluent, tablets operating with slow release and/or targeted release can be prepared.

Liquid preparations for oral administration can be made in the form of elixirs, syrups or suspensions, for example solutions containing from about 0.001 % to about 30% by weight of IFNτ, buffer and a mixture of sugar, ethanol, water, glycerol, propylene, glycol, amino acid, salt and possibly other additives of a conventional nature.

Another suitable formulation is a protective dosage form that protects the protein for survival in the stomach and intestines until released near the intestinal mucosa. Protective dosage forms for proteins are known in the art, and include enteric coatings and/or mucoadhesive polymer coatings. Exemplary mucoadhesive polymer formulations include ethyl cellulose, hydroxypropylmethylcellulose, Eudragit®, carboxyvinly polymer, carbomer, and the like. The IFNτ is preferably administered in a form that targets the intestinal tract of the patient, rather than the oral cavity. A dosage form designed for administration to the stomach via ingestion for delivery of IFNτ in an active form to the intestinal tract, and particularly to the small intestine, is contemplated. Alternatively, IFNτ can be co-administered with protease inhibitors, stabilized with polymeric materials, or encapsulated in a lipid or polymer particle to offer some protection from the stomach and/or intestinal environment.

C. Dosages

An orally-active IFNτ pharmaceutical composition is administered in a therapeutically-effective amount to an individual in need of treatment. The dose may vary considerably and is dependent on factors such as the nature and seriousness of the disorder, the age and the weight of the patient, other medications that the patient may be taking and the like. This amount or dosage is typically determined by the attending physician or other person of skill in the art. The dosage will typically be about 6×10⁸ to about 5×10¹² Units/day, more preferably about 0.5×10⁹ to about 1×10¹² Units/day, still more preferably about 1×10⁹ to about 1×10¹² Units/day. In one specific embodiment, IFN-τ is administered orally at a dosage of greater than about 5×10⁸ Units/day, more preferably at a dosage of 0.5×10⁹ Units/day or more, still more preferably at a dose of 1×10⁹ Units/day or more. The daily dosage may be administered all at once, or may be divided into two or more dosages to reach the daily dosage.

Disorders requiring a steady elevated level of IFNτ in plasma will benefit from administration as often as about every two to four hours, while other disorders, may be effectively treated by administering a therapeutically-effective dose at less frequent intervals, e.g., once a day, twice a day, three times a day, once every 48 hours, or once a week. The rate of administration of individual doses is typically adjusted by an attending physician or other person of skill in the art to enable administration of the lowest total dosage while alleviating the severity of the disease being treated. As discussed above, the method contemplates administering IFNτ orally at a first dose to a patient in need of treatment, and monitoring a biological marker to determine the individual patient response to the first dosage level. Monitoring can be readily done via a blood draw and analysis of a marker, such as IL-10 in the blood, using, for example, a ELISA or a radioimmunoassay kit.

Administration of IFNτ generally continues until a clinical endpoint is achieved. The clinical endpoint will vary according to the condition being treated, the severity of the condition, and to the patient's individual characteristics (age, weight, health). Clinical endpoints are readily determined by an attending doctor, nurse or other person of skill in the art and range from a temporary or permanent cessation of symptoms to resolution of the condition. The clinical endpoints typically include attenuation or lessening of the symptoms associated with the disease or biological confirmation that the disease severity has decreased, abated or its progression has stopped.

For example, in patients suffering from Alzheimer's disease, a decrease in endogenous amyloid plaques and/or neurofibrillary tangles in the brain of such patients may be observed. Decreases in memory loss, language deterioration, confusion, restlessness and mood swings; and improved ability to mentally manipulate visual information as determined by standard methods may also be observed.

In individuals afflicted with pulmonary fibrosis, suitable clinical endpoints include an improvement in the ability to breath, a decrease in the progression of the disease as evidenced by a reduction in amount of fibrous tissue in the lungs, and/or a decrease in lung inflammation. The effect of interferon-tau in ameliorating the decline in lung function, including the ability of the patient to breath after treatment compared to prior to treatment, may be measured by methods known to the skilled artisan, including measurement of the forced expiratory volume in one second (FEVI) as described, for example, in Pellegrino, R. and Brusasco, V., Eur. Respir. J. 10:543-549 (1997). A reduction in the increase in fibrous scar tissue in the lungs may be measured by methods known to the skilled artisan, including measurement of lung tissue hydroxyproline content, or other similar procedure known to the art, as described in Example 3. Decreases in lung inflammation may be observed by methods known to the art, including those discussed in Example 3.

In individuals with liver fibrosis, a suitable clinical endpoint includes a reduction in the increase in fibrous scar tissue in the liver as measured by methods known to the skilled artisan, including histological examination of liver tissue samples with scoring methods found in, for example, Desmet, V. J., et al., Hepatology 19:1513-1520 (1994) or Chevallier, M., et al., Hepatology 20:349-355 (1994). A decrease in serum hyaluronic acid levels and liver hydroxyproline content obtained by methods known to the art and described in Example 2 herein may also be observed. Additionally, a decrease in the serum level of various liver enzymes, including aspartate aminotransaminase (AST) and alanine aminotransaminase (ALT), measured by standard methods known to the art, may also be monitored.

In individuals who have experienced a stroke, a suitable clinical endpoint includes an increase in blood flow in the affected blood vessel as determined by computer tomographic methods as known in the art and as described, for example, in Nabavi, D. G., et al., Radiology 213:141-149 (1999). A further clinical endpoint includes a decrease in numbness in the face, arm or leg; or a decrease in the intensity of a headache associated with the stroke. Yet another clinical endpoint includes a decrease in the cell, tissue or organ damage or death due to the stroke. Such decrease in cell or tissue damage may be assessed by brain imaging techniques, including computer assisted tomography (CAT) scanning, magnetic resonance imaging methods, or similar methods known to the art.

In individuals who have experienced optic neuritis, a suitable clinical endpoint includes an improvement in vision, a stabilization of vision (i.e., no further decline in vision) or a decrease in the rate of decline of vision. Such clinical endpoints may be determined by the skilled artisan as known in the art.

In individuals with chronic obstructive pulmonary disease, a suitable clinical endpoint includes an improvement in lung function or an otherwise decrease in the extent of the blockage. These clinical endpoints may be determined by, for example, a pulmonary function test. In one type of pulmonary function test, the patient breathes into a spirometer, which is a mechanical device that records changes in lung size as air is inhaled and exhaled by the patient as a function of time.

In individuals with autism, the clinical endpoint will depend on the particular symptoms associated with the patient. The symptoms include abnormalities in social skills, speech, communication and repeated behaviors and routines. For example, autistic individuals may exhibit no speech, non-speech vocalizations, echolalia, confusion between the pronouns “I” and “You”, lack of eye contact, lack of response to people, and walking on tiptoes. Such abnormalities may be assessed by methods known to the skilled artisan.

In individuals with various autoimmune disorders, including diabetes, allergies, inflammatory bowel disease, psoriasis, arthritis, multiple sclerosis, uveitis, and anti-phospholipid syndrome, the clinical endpoint will depend on the particular disease or condition. For example, in individuals afflicted with anti-phospholipid syndrome, a suitable clinical endpoint includes reduction in the levels of antibodies directed against membrane anionic phospholipids, (e.g., anti-cardiolipin, anti-phosphatidylserine) or their associated plasma proteins (e.g., β-2-glycoprotein). Quantitation of such antibodies and or plasma proteins may be accomplished by routine methods known to the art, including by enzyme-linked inmmunosorbent assays (ELISA) as described, for example, in Pierangeli, S. S., et al., Thromb. Haemost. 74:1361-1367 (1995).

Yet another clinical endpoint in individuals with anti-phospholipid syndrome includes a decrease in vascular thrombosis, which may occur in a wide variety of tissue and/or organs. A decrease in vascular thrombosis may be observed by determining the blood flow in the affected tissue or organ by computer tomographic methods as described, for example, in Nabavi, D. G., et al., Radiology 213:141-149 (1999). A decrease in vascular thrombosis may also be histologically detected. Skin or other involved tissue may be analyzed. For example, biopsies from affected kidneys may show a reduction in glomerular and/or small arterial microthrombi. Additionally, the size of thrombi, as well as the rate of its disappearance, may be observed with fiber-optic devices used to transilluminate the vein and a trilocular stereoscopic operating microscope equipped with a closed-circuit video system, monitor and recorder as more fully described in Pierangeli, S. S., et al., Circulation 94:1746-1751 (1996) and Example 5 described herein.

A suitable clinical endpoint in diabetes includes the ability to control blood glucose levels either without insulin or with a decreased amount of insulin. A suitable clinical endpoint for various allergies include a decrease in various symptoms of allergies, including a decrease in the amount of wheezing, sneezing, watery eyes, nausea, vomiting or diarrhea associated with the allergic condition and may be determined by common methods in the art. A suitable clinical endpoint for inflammatory bowel disease, including Crohn's disease and ulcerative colitis, includes decreases in various symptoms of this disease, such as a decrease in the extent of diarrhea, a decrease in abdominal pain and/or cramping, decreased amount of blood in the stool, increased appetite, and decreased body temperature from fever associated with the disease. A suitable clinical endpoint with respect to psoriasis includes a decrease in the amount of the characteristic dry, red patches of skin covered with silvery scales or a decrease in the swelling or stiffness of affected joints. A suitable clinical endpoint in arthritis, such as found in rheumatoid arthritis, includes a decrease in the amount of pain and swelling in the affected joints, an increase in the range of motion of the patient, and an increase in the strength of the muscle attached to the affected joint. A suitable clinical endpoint for multiple sclerosis includes a decrease in attacks and a decrease in number of brain lesions. A suitable clinical endpoint for uveitis, including anterior uveitis, intermediate uveitis, posterior uveitis and diffuse uveitis, includes improvement in vision, including no further decline in vision, and a decrease in the amount of floaters. Decreased pain, redness and photophobia may be also be observed with anterior uveitis. A suitable clinical endpoint for allergies includes a decrease in allergic reactions, such as swelling in mucosal tissues, a decrease in inflammation in affected tissues and a decrease in overall IgE levels. Suitable clinical endpoints in organ transplantation include, for example, an increase in graft survival.

Suitable clinical endpoints in atherosclerosis will depend on the particular symptoms the patient exhibits. For example, the patient may exhibit decreased blood flow in one or more blood vessels. Accordingly, a suitable clinical endpoint includes increases in blood flow in the selected blood vessels. The rate of blood flow may be measured by methods known to the skilled artisan, including Laser Doppler Flowmetry (LDF), Magnetic Resonance Imaging (MRI), Positron Emission Tomography (PET), and Computed Tomography (CT) Imaging, including Single Photon Emission Computed Tomography (SPECT) [Leenders, K. L., et al. Brain 113:27-47 (1990); Sakai, F., et al. J. Cereb. Blood Flow Metab. 5:207-213 (1988); Rempp, K. A., et al, Radiology 193:637-641 (1994); Baird, A. E. and Warach, S., J. Cereb. Blood Flow Metab. 18:583-609 (1998); Danus, G., et al., Radiology 213:141-149 (1999); Calamante, F., et al., J. Cereb. Blood Flow Metab. 19:701-735 (1999); Ginsberg, M. D., et al., J. Cereb Blood Flow Metab. 2(1):89-98 (1982); Fukuda, O., Neurosurgery 36(2):358-364 (1995); Perez-Pinzon, et al., J. Neurolog. Sci. 153 (1):25-31 (1997); Borlongan, et al., Brain Res. 1010(1-2):108-116 (2004)]. If the patient presents with angina pectoris derived from the atherosclerosis, a decrease in pain associated with this condition may be observed. If the patient presents with peripheral vascular disease derived from the atherosclerosis, decreased claudication and decreased impotence may be observed. Other suitable clinical endpoints are known to the skilled artisan.

Once the desired clinical endpoint is achieved, treatment with IFNτ can cease, however a maintenance dose can be administered if desired or as necessary. Subsequently, the dosage or the frequency of administration, or both, may be reduced, as a function of the symptoms, to a level at which the clinical endpoint is maintained or the improved condition is retained.

D. Combination Therapy

In other aspects of the invention, oral administration of IFNτ in accordance with the invention may be used in combination with other therapies. The second therapeutic agent may be co-administered in a composition with interferon-tau or may be administered prior to or after administration of interferon-tau. The second therapeutic agent will be selected based on the particular disease or condition.

Alzheimer's disease may be benefited by administration of amyloid beta, neurotransmission enhancing drugs, including anti-cholinesterase inhibitors such as, for example, tacrine, donepezil, rivastigamine, metrifonate, epastigimine, nicotine, pyridostigimine, neostigimine, physostigimine, ambenomium chloride and Gingko biloba; substances that increase brain catecholamines and/or reduce oxidative damage to neurons, including selegiline and vitamin E; non-steroidal drugs having anti-inflammatory properties, including statins (the statins also have immunosuppressant properties), such as lovastatin, simvastatin, pravastatin, fluvastatin, atorvastatin, rosuvastatin, and cerivastatin; aminoarylcarboxylic acid derivatives, such as enfenamic acid, etofenamate, flufenamic acid, isonixin, meclofenamic acid and tolfenamic acid; arylacetic acid derivatives, such as aceclofenac, acemetacin, bromfenac, clopirac, etodolac, fentiazac, indomethacin, oxametacine, and tropesin; arylbutyric acid derivatives, such as bumadizon, butibufen, fenbufen and xenbucin; arylcarboxylic acid derivatives, such as clidanac, ketorolac, and tinoridine; arylpropionic acid derivatives, such as alminoprofen, carprofen, fenoprofen, ibuprofen, indoprofen, ketoprofen, naproxen, pirprofen and zaltoprofen; pyrazoles, such as difenamizole and epirizole; pyrazolones, such as apazone, benzpiperylon, feprazone, oxyphenbutazone, pipebuzone, ramifenazone, and thiazolinobutazone; salicylic acid derivatives, such as acetaminosalol, aspirin, balsalazide, diflunisal, gentisic acid, imidazole salicylate, olsalazine, parsalmide, salicylsulfuric acid, sodium salicylate and sulfasalzine; and thiazinecarboxyamides, such as ampiroxicam, droxicam, isoxicam, lornoxicam, piroxicam and tenoxicam; and cholinergic agonists, including xanomeline, milameline, AF 102B and memric.

Therapeutic agents that may be combined with interferon-tau treatment of liver fibrosis may be selected based on the cause of the fibrosis. For example, where liver fibrosis is caused by a hepatitis virus infection, such as hepatitis C infection, various anti-viral agents may be utilized, including monoclonal antibodies, such as palivizumab; peptidomimetics, such as amprenavir, indinavir, lopinavir, nelfinavir, ritonavir, and saquinavir; polynucleotides, such as ampligen and fomivirsen; purines/pyrimidones, such as abacavir, acyclovir, adefovir, cidofovir, cytarabine, didanosine, dideoxyadenosine, edoxudine, emtricitabine, famciclovir, floxuridine, ganciclovir, idoxuridine, inosine pranobex, lamivudine, MADU, penciclovir, sorivudine, stavudine, tenofovir, trifluridine, valacyclovir, valganciclovir, vidarabine, zalcitabine, and zidovudine; and sialic acid analogs, such as oseltamivir and zanamivir; and interferon-alpha, interferon-beta and interferon-gamma.

Where liver fibrosis is secondary to cancer, various anti-cellular proliferative agents may be utilized, including nitrogen mustards, ethyleneimines, methylmelamines, alkyl sulfonates, nitrosoureas, triazenes, folic acid analogs, pyrimidine analogs, purine analogs, vinca alkaloids, epipodophyllotoxins, antibiotics, enzymes, biological response modifiers (e.g., cytokines), platinum coordination complexes, anthracenedione, substituted ureas, methylhydrazine derivatives, adrenocortical suppressants, progestins, estrogens, antiestrogens, androgens, antiandrogens, and gonadotropin releasing hormone analogs. Representative drugs include, but are not limited to mechlorethamine, cyclophosphamide, ifosfamide, melphalan, chlorambucil, hexamethylmelamine, thiotepa, busulfan, carmustine, lomustine, semustine, streptozocin, dacarbazine, methotrexate, fluorouracil, floxuridine, cytarabine, mercaptopurine, thioguanine, pentostatin, vinblastine, vincristine, etoposide, teniposide, dactinomycin, daunorubicin, doxorubicin, bleomycin, plicamycin, mitomycin, asparaginase, interferon-alpha, cisplatin, carboplatin, mitoxantrone, hydroxyurea, procarbazine, mitotane, aminoglyethimide, prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, zidovudine (AZT), leucovorin, melphalan, dacarbazine, dipyridamole, and others. The skilled artisan can select other appropriate therapeutic agents where liver fibrosis is secondary to some other disease or condition.

Therapeutic agents that may be combined with interferon-tau treatment of pulmonary fibrosis include immunosuppressants, such as alemtuzumab, azathioprine, basiliximab, brequinar, cyclophosphamide, corticosteroids (prednisone, prednisolone, triamcinolone, betamethasone, dexamethasone, etc.), cyclosporine, gusperimus, 6-mercaptopurine, mizoribine, pimecrolimus, rapamycin, mycophenolate mofetil, antithymocyte globulin, muromonab-CD3 monoclonal antibody, mercaptopurine, mitoxantrone, glatiramer acetate (Copaxone®), interferon-gamma, interferon-beta (Avonex™, Betaseron™, Ribif™), daclizumab, methotrexate, sirolimus, tacrolimus, and others. Galtiramer acetate is a synthetic basic random copolymer composed of tyrosine, glutamate, alanine and lysine and has, for example, anti-inflammatory properties. When administered with IFNτ, glatiramer acetate could induce higher levels of IL-10 and TGF-beta (Soos, J M, et al., J Immunol 169:2231; 2002). In certain cases where the underlying disease which caused the pulmonary fibrosis is known, such as rheumatoid arthritis, and tuberculosis, therapeutic agents known to treat such diseases or conditions may be utilized. For example, antibiotic treatment may be utilized in tuberculosis; anti-inflammatory agents may be used in rheumatoid arthritis, including the non-steroidal anti-inflammatory drugs previously discussed herein; Cox-2 inhibitors, including valdecoxib, lumiracoxib and celecoxib; as well as immunosuppressants previously described herein, including methotrexate to take advantage of its, for example, immunosuppressant properties.

Therapeutic agents that may be combined with interferon-tau treatment of anti-phospholipid syndrome include anti-coagulants, such as unfractionated heparin, lovenox, acetylsalicylic acid, and coumadin; anti-malarials, including hydroxychloroquine, mefloquine, primaquine, proguanil, and doxycycline; corticosteroids, including prednisone, prednisolone, triamcinolone, betamethasone, and dexamethasone; and immunomodulatory agents, including intravenous immune globulins.

Therapeutic agents that may be combined with interferon-tau treatment of stroke include cerebral vasodilators, such as bencyclane, ciclonicate, cinnarizine, cyclandelate, diisopropylamine dichloroacetate, eburnamonine, fasudil, fenoxedil, flunarizine, ibudilast, ifenprodil, lomerizine, nafronyl, nicametate, nicergoline, nimodipine, papaverine, pentifylline, vincamine, vinpocetine and viquidil; coronary vasodilators, such as amotriphene, benfurodil hemisuccinate, benziodarone, chloracizine, chromonar, clobenfurol, clonitrate, cloricromen, dilazep, etafenone, fendiline, hexobendine, mannitol hexanitrate, nitroglycerin, pentrinitrol, perhexiline, propatyl nitrate, trapidil, trimetazidine and visnadine; and tissue plasminogen activator. Other suitable agents include auto-antigens, such as myelin oligodendrocyte glycoprotein (MOG) or peptides thereof, statins, and glatiramer acetate. The MOG is preferably a mammalian MOG, such as a human MOG. The nucleotide and amino acid sequence (SEQ ID NO:5) of human MOG is known in the art and can be found, for example, in the NIH Genbank database as accession number BC035938. Suitable MOG peptides include those which range from about 10 to about 150 amino acids long, typically about 30 to about 100 amino acids long and may further be about 10 to 50 amino acids long, and are effective in treating stroke patients. As one example, a peptide from amino acid 35 to amino acid 55 of MOG may be used. As another example, a peptide of MOG from amino acid 37 to amino acid 46 may be used. It is realized that MOG and peptides thereof from other mammalian species, including, for example, ovine, bovine and porcine, that function in treating stroke may be used in the methods herein. Therefore, applicable MOGs include those that have amino acid sequences that have at least about 70% identity, further at least about 80% identity, and further at least about 90% identity to the amino acid sequence of human MOG. Percent identity may be determined, for example, by comparing sequence information using the advanced BLAST computer program, including version 2.2.9, available from the National Institutes of Health, or may be determined using other similar computer programs. The BLAST program is based on the alignment method of Karlin and Altschul. Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Accordingly, administration of interferon-tau in combination with one or more of the above-recited therapeutic agents is included in the scope of the methods of the present invention.

Therapeutic agents that may be combined with interferon-tau treatment of optic neuritis include statins, and the immunosuppressants previously described herein, including statins, corticosteroids and glatiramer acetate.

Therapeutic agents that may be combined with interferon-tau treatment of chronic obstructive pulmonary disease include bronchodilators and corticosteroids. Suitable bronchodilators include, for example, ephedrine derivatives, such as albuterol, bambuterol, bitolterol, carbuterol, clenbuterol, dioxethedrine, ephedrine, fenoterol, isoetharine, mabuterol, pirbuterol, protokylol, rimiterol, salmeterol, soterenol, and tulobuterol; quaternary ammonium compounds, such as flutropium bromide, ipratropium bromide, oxitropium bromide and titropium bromide; and xanthine derivatives, such as acefylline, ambuphylline, aminophylline, bamifylline, doxofylline, etofylline, guathylline, theobromine and theophylline. Other suitable agents include statins, glatiramer acetate and corticosteroids previously described herein.

Therapeutic agents that may be combined with interferon-tau treatment of autism include serotonin uptake inhibitors, including femoxetine, fluoxetine, fluvoxamine, indalpine, indeloxazine hydrochloride, milnacipran, paroxetine, sertraline and zimeldine; anti-psychotic drugs, including the benzamides, such as amisulpride, nemonapride, remoxipride, sulpiride, and sultopride; the benzisoxazoles, such as iloperidone and risperidone; the butyrophenones, such as benperidol, bromperidol, droperidol, fluanisone, haloperidol, melperone, moperone, pipamperon, spiperone, timiperone, and trifluperidol; the phenothiazines, such as acetophenazine, butaperazine, carphenazine, chlorpromazine, clospirazine, cyamemazine, dixyrazine, fluphenazine, mepazine, and mesoridazine; and the thioxanthenes, such as chlorprothixine, clopenthixol, flupentioxl and thiothixen; and drugs having anti-inflammatory properties, including statins, glatiramer acetate and corticosteroids previously described herein.

Therapeutic agents that may be combined with interferon-tau treatment of diabetes include insulin, beta-cell associated auto-antigens, and heat shock proteins, including, for example, heat shock proteins 10, 22, 27, 60, 65, 70 and 90. The heat shock proteins are preferably mammalian heat shock proteins, such as human heat shock proteins, many of which may be purchased commercially or otherwise may be obtained by methods known to the skilled artisan.

Therapeutic agents that may be combined with interferon-tau treatment of atherosclerosis include the immunosuppressant agents previously described herein heat shock proteins, including, for example, heat shock proteins 10, 22, 27, 60, 65, 70 and 90. The heat shock proteins are preferably mammalian heat shock proteins, such as human heat shock proteins. Other suitable therapeutic agents include, for example, anti-platelet agents, such as aspirin, clopidogrel, dypridamole, ticlopidine, and sulfinpoyrazone; bile acid sequestrants, including colestipol hydrochloride, and cholestryamine resin; fibrinates (fibric acid derivatives), including gemfibrozil, fenofibrate, and clofibrate; 3-hydroxy-3-methylglutaryl coenzyme A (HMG-CoA) reductase inhibitors, such as lovastatin (Mevacor®), simvastatin (Zocor®; velostatin), pravastatin (Pravachol®), fluvastatin (Lescol®), atorvastatin (Lipitor®), rosuvastatin (Crestor®), and cerivastatin (Baycol®); an anti-hypertensive in cases of accompanying high blood pressure, including diuretics, such as organomercurials, including chlormerodrin, meralluride, mercaptomerin sodium and mersalyl; purines, including pamabrom, protheobromine, and theobromine; steroids, such as canrenone, oleandrin, and spironolactone, sulfonamide derivatives, such as acetazolamide, ambuside, azosemide, bumetanide, butazolamide, clofenamide, clopamide, disulfamide, furosemide, and torsemide; and thiazides, such as althiazide, benzthiazide, chlorothiazide, cyclopenthiazide, ethiazide, hydrochlorothiazide, indapamide, metolazone and teclothiazide; angiotensin converting enzyme inhibitors, such as alacepril, benazepril, captopril, ceronapril, cilazapril, delapril, enalapril, lisinopril, moveltipril, quinapril, ramipril and temocarpril; alpha-adrenergic agonists, such as adrafinil, adrenalone, amidephrine, apraclonidine, burdralazine, clonidine, cyclopentamine, ephedrine, fenoxazoline, mataraminol, methoxamine, midodrine, modafinil, octodrine, oxymetazline, pholedrine, rilmenidine and tyramine; beta-adrenergic blockers, such as acebutolol, alprenolol, amosulalol, arotinolol, atenolol, befunolol, betaxolol, bevantolol, bunitrolol, butidrine hydrochloride, carazolol, carteolol, dilevealol, indenolol, mepindolol, moprolol, nadoxolol, penbutolol, pindolol, propranolol, sulfinalol, tertatolol, and xibenolol; calcium channel blockers, including the arylalkylamines, such as bepridil, clentiazem, diltiazem, fendiline, gallopamil, mibefradil, prenylamine, semotiadil, terodiline, and verapamil; dihydropyridine derivatives, such as amlodipine, aranidipine, barnidipin, benidipin, manidipiine, nilvadipine and nitrendipine; and piperazine derivatives, such as cinnarizine, dotarizine, flunarizine, lidoflazine, and lomerizine; the immunosuppressants previously described herein and drugs having anti-inflammatory properties, including statins, glatiramer acetate and corticosteroids previously described herein.

Therapeutic agents that may be combined with interferon-tau treatment of allergies include allergy-inducing agents, including pollen, ovalbumin, food ingredients, such as milk, wheat, animal meat, including bovine, porcine or ovine-derived meat; vegetables, including carrots, and cruciferous vegetables such as broccoli, cabbage, cauliflower, brussels sprouts, and turnips; nuts, including peanuts, pistachios, cashews; and mites; endoplasmic reticulum degrading agents, including Der1 (from, for example, Saccharomyces cerevisiae, the sequence of which has been cloned described in Knop, M., et al., Embo J. 15(4) 753-763 (1996); glatiramer acetate and the statins and corticosteroids previously described herein. Additionally, Der1 proteins known in the art from other species may be used, as well as Der1 like proteins known to the art.

Therapeutic agents that may be combined with interferon-tau treatment of inflammatory bowel disease include infliximab (a monoclonal antibody against tumor necrosis-α factor, TNF-alpha factor) statins previously described herein, glatiramer acetate, anti-diarrheal agents, such as acetyltannic acid, alkofanone, bismuth subsalicylate, catechin, difenoxin, diphenoxylate, lidamidine, loperamide, racecadotril, trillium, uzarin and zaldaride; and antispasmodic agents, including belladonna, hyoscyamine, clidinium bromide, glycopyrrolate, dicyclomine hydrochloride, mebeverine, otilonium bromide, and cimetropium.

Therapeutic agents that may be combined with interferon-tau treatment of psoriasis may include topical or systemic agents. Suitable therapeutic agents include immunosuppressants, including monoclonal antibody against TNF-alpha factor, the statins and glatiramer acetate previously described herein, collagen, retinoids, anthralin, calpotriene, coal tar, salicylic acid, clobetasol propionate, alefacept, hydroxyurea, and etanercept.

Therapeutic agents that may be combined with interferon-tau treatment of arthritis include antigens, such as streptococcal cell wall; heat shock proteins, including those previously described herein such as heat shock protein 60; collagen, including type II collagen; non-steroidal anti-inflammatory agents previously described herein; monoclonal antibody against TNF-alpha factor, statins previously described herein, glatiramer acetate, methotrexate and COX-2 specific inhibitors previously described herein.

Therapeutic agents that may be combined with interferon-tau treatment of multiple sclerosis include myelin basic protein and peptides thereof, myelin oligodendrocyte glycoprotein and peptides thereof previously described herein, a monoclonal antibody against TNF-alpha factor previously described herein, glatiramer acetate and the previously described corticosteroids and statins. The myelin basic protein is preferably a mammalian myelin basic protein, such as a human myelin basic protein. The nucleotide and amino acid sequence (SEQ ID NO:6) of human myelin basic protein may be found, for example, in the NIH Genbank database as accession number NM_{—}002385. Suitable peptides of myelin basic protein include those from about 5 to about 30 amino acids long. Suitable peptides include the fragment from amino acid 1 to amino acid 11, which may include a modification at its 5′ end, such as an acetyl group. It is realized that myelin basic protein and peptides thereof from other mammalian species, including, for example, ovine, bovine and porcine, that function in treating multiple sclerosis may be used in the methods herein. Therefore, applicable myelin basic proteins include those that have amino acid sequences that have at least about 70% identity, further at least about 80% identity, and further at least about 90% identity to the amino acid sequence of human myelin basic protein.

Therapeutic agents that may be combined with interferon-tau treatment of uveitis include statins, glatiramer acetate, and other immunosuppressants previously described herein, including corticosteroids, and which may by administered systemically or in the form of drops for administration to the eye; uveitis inducing agents, including retinal S antigen (human retinal S antigen may be obtained as described in, for example, Beneski, D. A., et al., Inves. Opth. Vis. Sci. 25:686-690 (1984) and interstitial retinal binding protein [IRBP; the human sequence may be found in Liou, G. I., et al., J. Biol. Chem. 264 (14):8200-8206 (1989)]; and human leukocyte antigen (HLA)-binding peptides, including those derived from HLA-A, HLA-B, HLA-C, HLA-E and HLA-G known to the art and discussed in, for example, PCT international application numbers PCT/US02/2431 1 and PCT/GB98/03686; Braud, V. M., et al., Nature 391:795-799 (1998).

Therapeutic agents that may be combined with interferon-tau treatment of rejection from organ transplantation include immunosuppressants previously described herein, such as, for example, statins, glatiramer acetate, azathioprine, corticosteroids, or cyclophosphamide.

### IV. EXAMPLES

Reference will now be made to specific examples illustrating the invention described above. It is to be understood that the examples are provided to illustrate preferred embodiments and that no limitation to the scope of the invention is intended thereby.

Materials and Methods

A. Production of IFNτ

In one embodiment, a synthetic IFNτ gene was generated using standard molecular methods (Ausubel, et al., supra, 1988) by ligating oligonucleotides containing contiguous portions of a DNA sequence encoding the IFNτ amino acid sequence. The DNA sequence used may be either SEQ ID NO:1 or SEQ ID NO:4 or the sequence as shown in Imakawa, K. et al, Nature, 330:377-379, (1987). The resulting IFNτ polynucleotide coding sequence may span position 16 through 531: a coding sequence of 172 amino acids.

In one embodiment, the full length synthetic gene StuI/SStI fragment (540 bp) may be cloned into a modified pIN III omp-A expression vector and transformed into a competent SB221 strain of E. coli. For expression of the IFNτ protein, cells carrying the expression vector were grown in L-broth containing ampicillin to an OD (550 nm) of 0.1-1, induced with IPTG (isopropyl-1-thio-b-D-galactoside) for 3 hours and harvested by centrifugation. Soluble recombinant IFNτ may be liberated from the cells by sonication or osmotic fractionation.

For expression in yeast, the IFNτ gene may amplified using polymerase chain reaction (PCR; Mullis, K. B., U.S. Pat. No. 4,683,202, issued 28 Jul. 1987; Mullis, K. B., et al., U. S. Pat. No. 4,683,195, issued 28 Jul. 1987) with PCR primers containing StuI and SacI restriction sites at the 5′ and 3′ ends, respectively. The amplified fragments were digested with StuI and SacII and ligated into the SacII and SmaI sites of pBLUESCRIPT+(KS), generating pBSY-IFNτ. Plasmid pBSY-IFNτ was digested with SacII and EcoRV and the fragment containing the synthetic IFNτ gene was isolated. The yeast expression vector pBS24Ub (Ecker, D. J., et al., J. Biol. Chem. 264:7715-7719 (1989)) was digested with SalI. Blunt ends were generated using T4 DNA polymerase. The vector DNA was extracted with phenol and ethanol precipitated (Sambrook, J., et al., in MOLECULAR CLONING: A LABORATORY MANUAL, Second Edition, Cold Spring Harbor Laboratory, Cold Spring Harbor, N.Y. (1989)). The recovered plasmid was digested with SacII, purified by agarose gel electrophoresis, and ligated to the SacII-EcoRV fragment isolated from pBSY-IFNτ. The resulting recombinant plasmid was designated pBS24Ub-IFNτ.

The recombinant plasmid pBS24Ub-IFNτ was transformed into E. coli. Recombinant clones containing the IFNτ insert were isolated and identified by restriction enzyme analysis. IFNτ coding sequences were isolated from pBS24Ub-IFNτ and cloned into a Pichia pastoris vector containing the alcohol oxidase (AOX1) promoter (Invitrogen, San Diego, Calif.). The vector was then used to transform Pichia pastoris GS115 His⁻ host cells and protein was expressed following the manufacturer's instructions. The protein was secreted into the medium and purified by successive DEAE-cellulose and hydroxyapatite chromatography to electrophoretic homogeneity as determined by SDS-PAGE and silver staining.

B. Statistical Analyses

The results can be expressed as the mean±SEM. One way ANOVA may be used followed by Bonferroni's post hoc test.

### Example 1

### Effect of IFNτ Treatment on Alzheimer's Disease

This example shows how interferon-tau may be advantageous for treating Alzheimer's disease. A transgenic mouse model of Alzheimer's disease will be used. One group of the mice will receive interferon-tau treatment and the control group will receive none. It is expected that treatment of the transgenic mice with interferon-tau will exhibit a decreased plaque burden, a decrease in brain levels of amyloid beta peptide, and decreased microglial and astrocyte activation.

Materials and Methods

Animals

Ninety day old heterozygous male PDAPP transgenic mice with the APPV717F mutation [i.e., substitution of Phe for Val in the transmembrane domain of the amyloid precursor protein (APP) as discussed in Murrell, J. et al., Science 254(5028):97-99 (1991)] may be purchased from Taconic Farms (Germantown, N.Y.) and kept under standard laboratory conditions in accordance with the NIH Guide for the Care and Use of Laboratory Animals. The mice may be maintained on a 12 hour light and 12 hour dark cycle. The mice may be allowed free access to animal chow and tap water ad libitum

Measurement of Plaque Burden by Immunohistochemical and Histological Staining of Mouse Brains

Immunohistochemical and histological staining of mouse brains to determine plaque burden can be accomplished as described in, for example, Weiner, H. L., et al., Ann. Neurol. 48:567-579 (2000). Briefly, formalin-fixed brain tissue can be washed in Tris-buffered saline, dehydrated, and embedded in paraffin. Sagittal sections of brain tissue (e.g., ten micrometer sections) can be air dried and baked at 58° C. for one hour. Sections can be deparaffinized in Histoclear (National Diagnostics, Atlanta, Ga.) and rehydrated through graded ethanol to water.

Treatment

Mice may be treated with various dosages of interferon-tau (e.g., about 1×10⁵ U/day to about 5×10⁷ U/day) by oral (intragastric) administration.

### Example 2

### Effect of IFNτ Treatment on Liver Fibrosis

This example shows how interferon-tau treatment may be advantageous for treating liver fibrosis. Liver fibrosis will be induced in experimental animals by carbon tetrachloride treatment. One group of the carbon-tetrachloride treated animals will receive interferon-tau treatment and the control group will receive only vehicle. It is expected that the carbon-tetrachloride-treated rats will exhibit a decrease in the progression of the disease when treated with interferon-tau.

Materials and Methods

Sprague-Dawley rats weighing between 200 and 250 grams can be obtained from Taconic Farms (Germantown, N.Y.) and maintained as described in Example 1.

Induction of Hepatic Fibrosis

Liver fibrosis may be induced by intraperitoneal injections of carbon tetrachloride as described in Zhang, L. J., et al., World J. Gastroenterol. 10(1):77-81 (2004). Briefly, rats receive intraperitoneal injections of 50% carbon tetrachloride in saline at a dose of 2 ml/kg twice a week.

Histological Examination

Liver tissues may be fixed in formal and embedded in paraffin [Zhang, L. J., et al., World J. Gastroenterol. 10(1):77-81 (2004)]. Sections may be stained with hemotoxylin and eosin (HE) and examined under a light microscope. Stages of fibrosis may be assessed according to the criteria set forth in either Desmet, V. J., et al., Hepatology 19:1513-1520 (1994) or Chevallier, M., et al., Hepatology 20:349-355 (1994).

Biochemical Measurements

In order to determine the hydroxyproline content in liver, the liver may be first homogenized into power and hydrolyxed with 6M hydrochloric acid. [Weng, H. L., et al., World J. Gastroenterol. 7(1):42-48 (2001). The hydroxylproline content can be measured as described in Kivirikko, K. L., et al., Anal. Biochem 19:249-255 (1967).

Treatment

Rats may be treated with interferon-tau prior to, during or after administration of carbon tetrachloride. The rats can be treated with various dosages of interferon-tau (e.g., about 1×10⁵ U/day to about 5×10⁷ U/day). by oral (intragastric) administration. The control group will receive only vehicle.

### Example 3

### Effect of IFNτ Treatment on Pulmonary Fibrosis

This example shows how interferon-tau treatment may be advantageous for treating pulmonary fibrosis. Pulmonary fibrosis will be induced in experimental animals by bleomycin treatment. One group of the bleomycin-treated group will receive interferon-tau treatment and the control group will receive only vehicle. Indicators of inflammation, such as myeloperoxidase activity of a bronchoalveolar lavage and serum TNF-alpha will be monitored. Tissue hydroxyproline content will be measured to monitor the extent of fibrosis. It is expected that treatment of bleomycin-treated mice treated with interferon-tau will decrease the extent of fibrosis as determined by measuring the hydroxyproline content. It is also expected that interferon-tau will decrease the markers of inflammation (myeloperoxidase activity and amount of TNF-alpha as determined by TNF-alpha mRNA) in treated versus control mice.

Materials and Methods

Animals

Eight-week old male C57BL/6 mice may be purchased from Harlan (Indianapolis, Ind.) and maintained as described in Example 1.

Induction of Pulmonary Fibrosis

In order to induce pulmonary fibrosis, mice can receive a single dose of intratracheal bleomycin (0.8 mg/kg) [Arai, T. et al., Am J. Physiol. Lung Cell. Mol. Physiol. 278:L914-L922 (2000)]. Control mice are treated with vehicle. The mice may be sacrificed 7 days later (to examine inflammatory indicators) or 21 days later (to examine fibrotic indicators) by inhalation of sevoflurane. The lungs may then be excised, and blood may be obtained from the right ventricle. A bronchoalveolar lavage may be performed by instilling 1 ml of isotonic saline and withdrawn from the lungs with an intratracheal cannula.

Quantitation of Myeloperoxidase Activity

The myeloperoxidase activity of the bronchoalveolar lavage may be determined as known in the art [Arai, T. et al., Am J. Physiol. Lung Cell. Mol. Physiol. 278:L914-L922 (2000)]. Briefly, the bronchoalveolar lavage can be centrifuged at 400 g for 5 minutes. The cell pellet can be resuspended in 0.1 M K₂HPO₄ buffer and sonicated for 90 seconds. The supernatant can be mixed, after centrifugation at 12,000 g for 10 minutes, with 0.3 ml of Hanks' BSS containing 0.25% bovine serum albumin, 0.25 ml of 0.1 M K₂HPO₄ (pH 7.0), 0.05 ml of 1.25 mg/ml o-dianisidine (Sigma, St. Louis, Mo.), and 0.05 ml of 0.05% H₂O₂ and incubated for 10 minutes at 25° C. The reaction can be terminated by adding 0.5 ml of 1% NaN₃, and absorbance can be measured at 460 nm.

Treatment

Bleomycin treated mice may be treated with interferon-tau at the same time that bleomycin is introduced, or prior to or after administration. The mice may be treated with various dosages of interferon-tau (e.g., about 1×10⁵ U/day to about 5×10⁷ U/day) by oral (intragastric) administration. Bleomycin-treated mice in the control group will receive only vehicle.

### Example 4

### Effect of IFNτ Treatment on Stroke

This example shows how interferon-tau may be advantageous for treating stroke. A stroke will be induced in experimental animals by occlusion of the middle cerebral artery with an intraluminal suture. The stroke-induced group will receive interferon-tau treatment and the control group will receive only vehicle. The protective effects of interferon-tau treatment can be assessed by determining brain infarct size, measurement of apoptotic cell nuclei in the brain and assessment of behavior using a scale known in the art. It is expected that mice subjected to stroke and treated with interferon-tau will exhibit decreased brain infract size, decreased cerebral apoptotic nucleic and improved neurological function when compared to control mice.

Materials and Methods

Animals

Male Sprague Dawley neonatal rats (9 to 11 days of age) may be obtained from Taconic Farms (Germantown, N.Y.). The mice may be maintained as described in Example 1.

Middle Cerebral Artery Occlusion Model

Cerebral ischemia can be induced in the Sprague Dawley rats using an occluding intraluminal suture as described in Maier, C. M., et al., J. Neurosurg 94:90-96 (2001) and Bright, R., et al., J. Neurosci. 24(31):6880-6888 (2004). Briefly, after anesthetizing the animal with isoflurane, the tip of an uncoated 30 mm long segment of 3-0 nylon suture is rounded by flame. The suture is inserted into the stump of the external carotid artery and advanced into the internal carotid artery about 19 to about 20 mm from the bifuracation to occlude the ostium of the middle cerebral artery. After 2 hours of ischemia, the suture can be removed and the animal can be allowed to recover.

Assessment of Behavior After Middle Cerebral Artery Occlusion

Behavior can be assessed at 24 hours after reperfusion using a scale of 1-4 described in Bright, R., et al., J. Neurosci. 24(31):6880-6888 (2004): grade 1, normal posture, spontaneous movement in any direction; grade 2, unilateral paw extension when lifted by the tail; grade 3, both paw extension and circuling pattern when spontaneously walking; grade 4, abnormal posture, paw extension, circuling pattern, can not walk spontaneously.

Measurement of Apoptotic Cell Nucleic

Apoptotic cell nucleic may be measured by terminal deoxynucleotidyl transferase-mediated biotinylated UTP nicke end labeling (TUNEL) as described in Bright, R., et al., J. Neurosci. 24(31):6880-6888 (2004). Briefly, animals subjected to a 2 hour middle cerebral artery occlusion can be sacrificed after 72 hours of reperfusion and transcardially perfused with normal saline followed by 4% paraformladehyde. Brains can be isolated and fixed overnight in 4% paraformaldehyde, immersed in 30% sucrose for about 2 to about 3 days, and snap frozen in OCT cyroprotectant (Tissue-Tek, Miles, Eklkart, Ind.).

Coronal slices (16 μm) can be taken from equivalent midbrain regions in each brain, with each slice separated by at least 48 μm. Slides can be stained and images can be taken from four defined regions in the ipsilateral cortex of each slice.

Treatment

Rats may be treated with interferon-tau prior to, during or after induction of ischemia. The rats can be treated with various dosages of interferon-tau (e.g., about 1×10⁵ U/day to about 5×10⁷ U/day).by oral (intragastric) administration. The control group will receive only vehicle.

### Example 5

### Effect of IFNτ Treatment on Anti-Phospholipid Syndrome (APS)

This example shows how interferon-tau may be advantageous for treating APS. APS will be induced in experimental animals by injection of human immunoglobulin G (IgG) anti-cardiolipin antibodies. One group of the antibody-treated group will receive interferon-tau treatment and the control group will received only vehicle. One femoral vein will be subjected to an injury which induces a thrombus and the size of the thrombus that forms, as well as the time of formation and disappearance of the thrombus, in control and interferon-tau treated animals will be used as an indicator of the effectiveness of treatment. It is expected that treatment of antibody-treated mice with interferon-tau will decrease the size of the initial thrombus that forms, increase the time the thrombus takes to form and decreases the time it takes the thrombus to disappear.

Materials and Methods

Animals.

Normal male CD-1® Nude Mice (Crl:CD-1®-nuBR;outbred), developed from the transfer of the nude gene to a CD-1 mouse through a series of crosses and backcrosses, which is T-cell deficient, may be purchased from Charles River Laboratories (Wilmington, Mass.) and maintained as described in Example 1.

Isolation of IgG from an Anti-Phospholipid Patient

IgG may be isolated from the serum of a patient with APS by protein G sepharose chromatography. [Pierangeli, S. S., et al., Circulation 94:1746-1751 (1996).] The purity of the IgG may be determined by observing a single band of 150 kD by sodium dodecyl sulfate-polyacrylamide gel electrophoresis (SDS-PAGE). Protein concentration may be determined by the Lowry method. After the protein concentration is adjusted with sterile saline solution, the solutions may be filter sterilized before injecting the animals.

Immunization of Mice

Mice may be immunized on days 1, 7, 14 and 21 by subcutaneous injection with 150 ug of IgG-APS in adjuvant (Adju-Prime, Pierce Chemical Co.) [Pierangeli, S. S., et al., Circulation 94:1746-1751 (1996).]. A specimen of blood can be drawn from each animal at weekly intervals to test for the presence of mouse anti-cardiolipin antibodies. Mouse anti-cardiolipin antibodies (IgG and IgM) may be determined by an enzyme-linked immunosorbent assay (ELISA) as known in the art and as described, for example, in Pierangeli, S. S., et al., Thromb. Haemost. 74:1361-1367 (1995). Alkaline phosphatase anti-mouse IgG and anti-mouse IgM may be used as secondary antibodies in the ELISA protocol. The color reaction may be stopped when a positive control (e.g., of about 100 G phospholipids units) reaches 1.0 OD units (typically in about 20 or 30 minutes).

Thrombi Induction

After the mice have been found to produce relatively high levels of anti-cardiolipin antibodies (e.g., about 0.8 OD units; typically occurs after about two weeks after immunization), thrombi are induced by a pinch injury as more fully described in Pierangeli, S. S., et al., Circulation 94:1746-1751 (1996). Briefly, animals may be treated with anesthetic (sodium pentobarbital, 60 nmg/kg intraperitoneally) prior to making a longitudinal incision in the right groin of the mice, extending to the kneed. The right femoral vein is dissected free and a standardized thrombogenic injury may be produced in the vein by bringing together forceps until the flat circular opposing surfaces (may be about 0.1 mm in diameter) meet to produce the pinch injury.

A fiber-optic device may be used to transilluminate the vein and a trilocular stereoscopic operating microscope (ERNST, Leitz GMBH, Wetzlar) equipped with a closed-circuit video system (NEC-NC-A/CCD Camera, NEC, USA, Inc.), a Panasonic 12″ color monitor and U-Matic V-5800 recorder (Sony Corp.) can be used to visualize and measure the size of the thrombus and its rate of appearance and disappearance Pierangeli, S. S., et al., Circulation 94:1746-1751 (1996).

Treatment

Mice may be treated with interferon-tau after injection with the antibodies from APS patients. The mice can be treated with various dosages of interferon-tau (e.g., about 1×10⁵ U/day to about 5×10⁷ U/day) by oral (intragastric) administration. The control group will receive only vehicle.

### Example 6

### Effect of IFNτ Treatment on Optic Neuritis

This example shows how interferon-tau may be advantageous for treating optic neuritis. Experimental allergic encephalitis (EAE), an animal model of optic neuritis, will be induced in test animals. The EAE-induced group will receive interferon-tau treatment and the control group will receive only vehicle. The protective effects of interferon-tau treatment can be assessed by determining the extent of axonal demyelination. It is expected that EAE-induced mice treated with interferon-tau will exhibit decreased axonal demyelination when compared to control mice.

Materials and Methods

Animals

SJL/J mice may be purchased from the Jackson Laboratory (Bar Harbor, Me.) and maintained as described in Example 1.

Induction of EAE

EAE can be induced in test mice by sensitization with homologous spinal cord emulsion in complete Freund;s adjuvant that can be injected subdermally into the nuchal area as described, for example, in Guy, J., et al., Proc. Natl. Acad. Sci. U.S.A. 95:13847-13852 (1998).

Analysis of Axonal Demyelination

The extent of demyleination may be quantitated by threshold measurements of the myelin sheaths derived from axonal transmission electron micrographs for each optic nerve as described in Guy, J., et al., Proc. Natl. Acad. Sci. U.S.A. 95:13847-13852 (1998).

Briefly, mice may be sacrificed and perfused by cardiac puncture with fixative consisting of 4% paraformaldehyde in 0.1 M PBS buffer (pH 7.4) The eyes with attached optic nerves can be dissected out and tissue specimens will be postfixed in 5% acrolein, 0.1 M sodium cacodylate-HCL buffer (pH 7.4) and 7% sucrose and dehydrated through an ethanol series and embedded in LR White that can be polymerized at 50° C. overnight. Ultrathin sections (e.g., about 90 mm) can be placed on nickel grids for immunocytochemistry. Nonspecific binding of antibodies can be blocked by floating the grids on 2% teleost gelatin and 2% nonfat dry milk in 0.01 M TBS (pH 7.2) with Tween 20 for 30 minutes. The grids can then be reacted with rabbit anti-albumin antibodies, washed and then reacted with secondary goat anti-rabbit igG antibodies conjugated to 10 nm gold for about 1 hour at room temperature. Grids can then be rinsed in deionized water. Immunolabeled specimens can be photographed by transmission electron microscopy without poststaining.

Treatment

Mice may be treated with interferon-tau prior to injection with the spinal chord emulsion, after injection with spinal chord emulsion or after optic neuritis is induced. The mice can be treated with various dosages of interferon-tau (e.g., about 1×10⁵ U/day to about 5×10⁷ U/day).by oral (intragastric) administration. The control group will receive only vehicle.

### Example 7

### Effect of IFNτ Treatment on Chronic Obstructive Pulmonary Disease

This example shows how interferon-tau may be advantageous for treating chronic obstructive pulmonary disease. Patients diagnosed with a chronic obstructive pulmonary disease, including chronic bronchitis and emphysema, will be treated with interferon-tau. The protective effects of interferon-tau treatment can be assessed by determining improvements in lung function. It is expected that patients treated with interferon-tau will exhibit improved lung function.

Materials and Methods

Human subjects who have been diagnosed with a chronic obstructive pulmonary disease can be treated with more than 0.5×10⁹ antiviral units of interferon-tau daily. Improvements in lung function can be assessed with a spirometer as known in the art.

### Example 8

### Effect of IFNτ Treatment on Autism

This example shows how interferon-tau may be advantageous for treating autism. Patients diagnosed with autism will be treated with interferon-tau. The advantageous effects of interferon-tau treatment can be assessed by determining alterations in the patient's pre-existing behavior. It is expected that patients treated with interferon-tau will exhibit positive changes in behavior.

Materials and Methods

Human subjects who have been diagnosed with autism can be treated with more than 0.5×10⁹ antiviral units of interferon-tau. Alterations in behavior, including improvements in social skills, speech, communication and/or repeated behaviors and routines may be assessed by methods known to the skilled artisan.

### Example 9

### Effect of IFNτ Treatment on an Autoimmune Disorder

This example shows how interferon-tau may be advantageous for treating and/or preventing collagen-induced arthritis (CIA) in mice. CIA will be induced in Balb C mice by injection of type II collagen. Prior to induction, mice will receive interferon-tau treatment. The advantageous effects of interferon-tau treatment can be assessed by determining the extent of development of CIA in the mice. It is expected that mice treated with interferon-tau will exhibit decrease disease incidence.

Materials and Methods

Induction of CIA

Inhibition of development of CIA in Balb C mice may be performed by injecting Balb C mice with a single dose of interferon-tau on the day of immunization with chicken type II collagen for induction of CIA. Collagen can be emulsified in complete Freund's adjuvant with H37Ra and may be injected on either side of the base of the tail. On the day of immunization and 48 hours later, Pertussis toxin may also be injected.

Interferon-Tau Production

Recombinant ovine interferon-tau may be expressed in Pichia pastoris using a synthetic gene construct. The protein can be secreted into the medium and purified by successive DEAE-cellulose and hydroxyapatite chromatography to electrophoretic homogeneity as determined by SDS-PAGE and silver staining.

Monitoring of Disease Development

Mice can be examined daily for signs of CIA. The severity of the disease may be graded as an arthritic index on the following scale: 1, redness of one toe; 2, redness and swelling of one toe; 3, deformation of one toe; 4 for each additional toe, the index number is added to the index.

Treatment

Mice may be treated with interferon-tau after the transplantation procedure. The mice may be treated with various dosages of interferon-tau (e.g., about 1×10⁵ U/day to about 5×10⁷ U/day) by oral (intragastric) administration. Mice in the control group will receive only vehicle.

### Example 10

### Effect of IFNτ Treatment on Atherosclerosis

This example shows how interferon-tau may be advantageous for treating atherosclerosis. Atherosclerosis will be induced in low density receptor (LDL)-deficient mice by feeding them a high cholesterol diet. One group of mice will receive interferon-tau and the control group will receive only vehicle. The protective effects of interferon-tau treatment can be assessed by determining the extent of atherosclerotic plaques that develop in the mice. It is expected that mice treated with interferon-tau will exhibit decreased atherosclerotic plaques when compared to control mice.

Materials and Methods

Animals

LDL receptor deficient mice may be purchased from Jackson Laboratories and maintained as described in Example 1.

Induction of Atherosclerosis

Atherosclerosis may be induced in the mice by feeding them a diet high in fat and cholesterol as described in Lichtman, A. H., et al., Arterioscler. Thromb. Vasc. Biol. 19:1938-1934 (1999).

Quantitation of Atherosclerotic Plaques

The surface area of the aorta occupied by atherosclerotic lesions may be quantified by en face oil red O staining as more fully described in Lichtman, A. H., et al., Arterioscler. Thromb. Vasc. Biol. 19:1938-1934 (1999). Briefly, mice may be sacrificed after 12 weeks on the particular diet by ether inhalation. After perfusion of the left ventricle and arterial tree with PBS, the entire aorta attached to the heart can be dissected and placed in formaldehyde overnight. The aorta can then be stained with oil red O, the adventitial fat is removed and the aorta can then be opened longitudinally, pinned en face on a black silicone-covered dish and photographed while immersed in PBS. Slides can then be scanned into a computer and the percent surface area occupied by oil red O-stained lesions can be determined by using image analysis software (NIH Image).

Treatment

Mice may be treated with interferon-tau when they are on cholesterol-rich diets. The mice may be treated with various dosages of interferon-tau (e.g., about 1×10⁵ U/day to about 5×10⁷ U/day) by oral (intragastric) administration. Mice in the control group will receive only vehicle.

### Example 11

### Effect of IFNτ Treatment on Organ Transplant Rejection

This example shows how interferon-tau may be advantageous for preventing or otherwise ameliorating organ transplant rejection. Kidney transplantation in a rat model will be performed. One group of rats will be treated with interferon-tau and the control group will receive only vehicle. The effect of interferon-tau in allograft rejection, as measured by renal allograft survival and the presence of selected immune system cells, will be examined. Treatment with interferon-tau is expected to increase renal allograft survival and decrease the presence of selected immune systems cells in the graft.

Materials and Methods

Animals

Inbred Fisher 344 (F344) and Lewis rats may be purchased from Charles River Italia (Calco, Italy). Lewis rats can be the recipients and the Fisher rats can be the donors. The animals may be maintained as described in Example 1.

Kidney Transplantation

Kidney transplantation may be accomplished as described in Noris, M., et al., J. Am. Soc. Nephrol. 12:1937-1946 (2001). Briefly, the left donor kidney may be removed and positioned orthotopically into the recipient whose renal vessels have been isolated and clamped and whose left native kidney has been removed. End-to-end anastomosis of the renal artery, vein and ureter can be performed using 10-O Prolen sutures. The right native kidney can be removed on the 11^{th} postoperative day. Complete allograft failure can be defined as death of the animal because the animals are dependent on the transplanted kidney function.

Immunhistochemical Analysis

Immunohistochemical analysis for selected immune system cells may be performed as described in Noris, M., et al., J. Am. Soc. Nephrol. 12:1937-1946 (2001). Briefly, mouse monoclonal antibodies can be used for the detection of the following antigens: 1) ED1 antigen; 2) a rat MHC class II antigen monomorphic determinant; 3) CD4 cell surface glycoprotein; 4) rat CD8 cell surface glycoprotein; 5) rat dendritic cells-restricted antigen. All antigens can be analyzed by an indirect immunofluorescence technique.

Treatment

Rats may be treated with interferon-tau after the transplantation procedure. The rats may be treated with various dosages of interferon-tau (e.g., about 1×10⁵ U/day to about 5×10⁷ U/day) by oral (intragastric) administration. Rats in the control group will receive only vehicle.

The invention has been described above in detail, with specific reference to its preferred embodiments. It will be understood, however, that a variety of modifications and additions can be made to the invention disclosed without departing from the spirit and scope of the invention. Such modifications and additions are desired to be protected. In addition, all references cited herein are indicative of the level of skill in the art and are hereby incorporated by reference in their entirety.

1. A method of treating a disease or condition responsive to interleukin-10 therapy in a mammal, comprising:
orally administering a daily dosage of greater than about 5×10⁸ Units of interferon tau to the mammal.

2. The method of claim 1, wherein said disease or condition is Alzheimer's disease, liver fibrosis, pulmonary fibrosis, autism, chronic obstructive pulmonary disease, rejection from organ transplant, anti-phospholipid syndrome, atherosclerosis or stroke.

3. The method of claim 1, wherein said interferon-tau is ovine interferon-tau or bovine interferon-tau.

4. The method of claim 3, wherein said ovine interferon-tau has an amino acid sequence set forth in SEQ ID NO:2 or SEQ ID NO:3.

5. The method of claim 1, wherein said oral administration is to the intestinal tract of the mammal.

6. The method of claim 1, wherein said daily dosage is sufficient to produce an initial measurable increase in the mammal's blood IL-10 level, relative to the blood IL-10 level in the mammal in the absence of interferon-tau administration.

7. The method of claim 6, further comprising continuing to orally administer interferon-tau to the mammal at least several times per week, independent of changes in the mammal's blood IL-10 level.

8. The method of claim 1, further comprising administering a second therapeutic agent to said mammal.

9. The method of claim 8, wherein said second therapeutic agent is co-administered in a composition with said interferon-tau.

10. The method of claim 8, wherein said disease or condition is Alzheimer's disease and said second therapeutic agent is amyloid beta, a neurotransmission enhancing drug, or an anti-inflammatory drug.

11. The method of claim 10, wherein said anti-inflammatory drug is a statin, glatiramer acetate, azathioprine, a corticosteroid, or cyclophosphamide.

12. The method of claim 8, wherein said disease or condition is liver fibrosis and said second therapeutic agent is an immunosuppressant, anti-viral agent or an anti-cellular proliferative agent.

13. The method of claim 12, wherein said immunosuppressant is a statin, glatiramer acetate, azathioprine, a corticosteroid, or cyclophosphamide.

14. The method of claim 8, wherein said disease or condition is pulmonary fibrosis and said second therapeutic agent is an immunosuppressant, an antibiotic or an anti-inflammatory agent.

15. The method of claim 14, wherein said immunosuppressant is a statin, glatiramer acetate, azathioprine, a corticosteroid, or cyclophosphamide.

16. The method of claim 15, wherein said corticosteroid is prednisone, prednisolone, triamcinolone, betamethasone, or dexamethasone.

17. The method of claim 8, wherein said disease or condition is autism and said second therapeutic agent is a serotonin uptake inhibitor, an anti-psychotic drug, a statin, glatiramer acetate, azathioprine, a corticosteroid, or cyclophosphamide.

18. The method of claim 8, wherein said disease or condition is chronic obstructive pulmonary disease and said second therapeutic agent is a bronchodilator, a statin, glatiramer acetate, azathioprine, a corticosteroid, or cyclophosphamide.

19. The method of claim 8, wherein said disease or condition is rejection from organ transplant and said second therapeutic agent is an immunosuppressant.

20. The method of claim 19, wherein said immunosuppressant is a statin, glatiramer acetate, azathioprine, a corticosteroid, or cyclophosphamide.

21. The method of claim 8, wherein said disease or condition is anti-phospholipid syndrome and said second therapeutic agent is an anti-coagulant, an anti-malarial, a corticosteroid, or an immunomodulatory agent.

22. The method of claim 21, wherein said anti-coagulant is unfractionated heparin, lovenox, acetylsalicylic acid, or coumadin; said anti-malarial is hydroxychloroquine; said corticosteroid is prednisone, prednisolone, triamcinolone, betamethasone, or dexamethasone; and said immunomodulatory agent is intravenous immune globulins.

23. The method of claim 8, wherein said disease or condition is atherosclerosis and said second therapeutic agent is a heat shock protein, an anti-platelet agent, a beta-adrenergic blocker, a calcium channel blocker, a statin, glatiramer acetate, azathioprine, a corticosteroid, or cyclophosphamide.

24. The method of claim 8, wherein said disease or condition is stroke and said second therapeutic agent is myelin oligodendrocyte glycoprotein or a peptide thereof, myelin basic protein or a peptide thereof, a statin, or glatiramer acetate.

25. The method of claim 1, wherein said disease or condition is type I diabetes mellitus, rheumatoid arthritis, psoriasis, multiple sclerosis, inflammatory bowel disease, an allergy, optic neuritis, or uveitis.

26. The method of claim 8, wherein said disease or condition is type I diabetes mellitus and said second therapeutic agent is insulin, a beta cell associated antigen or a heat shock protein.

27. The method of claim 8, wherein said disease or condition is rheumatoid arthritis and said second therapeutic agent is a monoclonal antibody against TNF-α factor, collagen, a statin, glatiramer acetate, a COX2-specific inhibitor, methotrexate or cyclosporine.

28. The method of claim 8, wherein said disease or condition is psoriasis and said second therapeutic agent is collagen, a retinoid, anthralin, calpotriene, coal tar, salicylic acid, or an immunosuppressant.

29. The method of claim 28, wherein said immunosuppressant is a statin, glatiramer acetate, a corticosteroid or a monoclonal antibody against TNF-alpha factor.

30. The method of claim 8, wherein said disease or condition is multiple sclerosis and said second therapeutic agent is myelin basic protein or a peptide thereof, myelin oligodendrocyte glycoprotein or a peptide hereof, a statin, glatiramer acetate, a monoclonal antibody against TNF-alpha factor or an immunosuppressant.

31. The method of claim 30, wherein said immunosuppressant is prednisone, prednisolone, triamcinolone, betamethasone, or dexamethasone.

32. The method of claim 8, wherein said disease or condition is inflammatory bowel disease and said second therapeutic agent is a statin, glatiramer acetate, a corticosteroid, or a monoclonal antibody against TNF-alpha factor.

33. The method of claim 8, wherein said disease or condition is an allergy and said second therapeutic agent is an allergy-inducing agent, a statin, glatiramer acetate or a corticosteroid.

34. The method of claim 33, wherein said allergy-inducing agent is pollen, ovalbumin, or a food ingredient.

35. The method of claim 34, wherein said food ingredient is milk, wheat, a nut; an animal meat or a vegetable.

36. The method of claim 8, wherein said disease or condition is optic neuritis and said second therapeutic agent is a statin, glatiramer acetate, azathioprine, a corticosteroid, or cyclophosphamide.

37. The method of claim 8, wherein said disease or condition is uveitis and said second therapeutic agent is a statin, glatiramer acetate or a corticosteroid.
